(19) United States
(12) Patent Application Publication
(10) Pub. No.: US 2021/0153783 A1
(43) Pub. Date: May 27, 2021

(54) WEARABLE DEVICE FOR IN-SITU ANALYSIS OF HORMONES

(71) Applicant: THE REGENTS OF THE UNIVERSITY OF CALIFORNIA, Oakland, CA (US)

(72) Inventors: Sam EMAMINEJAD, Los Angeles, CA (US); Sanaz PILEHVAR, Los Angeles, CA (US)

(73) Assignee: THE REGENTS OF THE UNIVERSITY OF CALIFORNIA, Oakland, CA (US)

(21) Appl. No.: 17/048,545

(22) PCT Filed: Apr. 17, 2019
(86) PCT No.: PCT/US2019/027923

§ 371 Date: Oct. 16, 2020

Related U.S. Application Data
(60) Provisional application No. 62/660,144, filed on Apr. 19, 2018.

(51) Int. Cl. A61B 5/1477; A61B 5/145; A61B 5/00; G01N 27/327

(52) U.S. Cl. CPC A61B 5/1477; A61B 5/14521; A61B 5/6833; A61B 10/0064; A61B 5/14532; A61B 5/14546; G01N 27/3276

(57) ABSTRACT

A wearable device for biofluid analysis includes a set of sensing electrodes, and the set of sensing electrodes includes a working electrode which includes: (1) abase electrode including a sensing surface; (2) capture probes immobilized on the sensing surface; and (3) a protective layer e disposed on the sensing surface and including a redox couple within the protective layer.

### CROSS-REFERENCE TO RELATED APPLICATIONS

This application claims the benefit of U.S. Provisional Application No. 62/660,144, filed Apr. 19, 2018, the contents of which are incorporated herein by reference in their entirety.

### TECHNICAL FIELD

This disclosure generally relates to a device and a method for biofluid analysis.

### BACKGROUND

Unlocking the full potential of sweat-based health monitoring involves accessing low-abundant (e.g., nanomolar (nM) to picomolar (pM) level) sweat analytes that are of significant value in molecular diagnostics. These analytes include hormones (e.g., cortisol), which are informative of immune function, stress and depression. However, demonstrated on-body analyte sensing (e.g., in sweat) has been constrained to high-concentration (e.g., millimolar (mM) to micromolar (μM) level) analytes. That is due to the constraints of sensing interfaces in sensitivity and the lack of suitable in-situ labeling strategies.

It is against this background that a need arose to develop the embodiments described herein.

### SUMMARY

Some embodiments are directed to a wearable device and methodology for the analysis of hormones such as cortisol. Demonstration is made of the device and methodology in the context of cortisol detection. The methodology is based on the label-free and direct electrochemical detection of aptamer-cortisol interactions by way of electrochemical impedance spectroscopy (EIS). A sensing interface includes cortisol-recognizing DNA aptamers as capture probes or receptors, which are covalently immobilized on a surface of a gold electrode by way of thiol-gold chemical bonding. The detection principle is based on changes of an interfacial resistance of the electrode, which can be measured in the presence of a reversible redox couple or probe [Fe(CN)₆]³⁻/[Fe(CN)₆]⁴⁻ using electrochemical impedance measurements. In this way, the surface of the electrode is partially blocked because of the formation of aptamer-cortisol complexes, resulting in the detection of an increase of the interfacial electron-transfer resistance. Electrochemical impedance measurements can lead to highly sensitive detection of a low concentration of cortisol. To realize this sensitivity, the methodology resolves a number challenges related to the [Fe(CN)₆]³⁻/[Fe(CN)₆]⁴⁻ redox couple in terms of repeatability and long-term use. For example, an etching mechanism of the surface of the gold electrode via pinhole regions by CN⁻ released from the [Fe(CN)₆]³⁻/[Fe(CN)₆]⁴⁻ redox couple can lead to the gradual removal of self-assembled monolayer of DNA aptamers. To this end, the improved sensing interface includes a protective layer (e.g., an absorbent pad) treated with the redox couple, which can be mounted on the surface of the electrode, protecting the electrode from direct exposure to an CN⁻-containing solution and enhanced stability of the receptor layer. In this way, electrochemical impedance response can be measured for different concentrations of cortisol in the presence of the redox couple.

Although some embodiments are explained in the context of sweat analysis, a non-invasive monitoring device and methodology can be used to analyze a variety of biofluids, including interstitial fluids and saliva, and can be used for a variety of applications, including stress and depression monitoring.

In some embodiments, a wearable device for biofluid analysis includes a set of sensing electrodes, and the set of sensing electrodes includes a working electrode which includes: (1) a base electrode including a sensing surface; (2) capture probes immobilized on the sensing surface; and (3) a protective layer disposed on the sensing surface and including a redox couple within the protective layer.

In some embodiments, a method for biofluid analysis includes: (1) providing an electrode and capture probes immobilized on a sensing surface of the electrode; (2) providing a redox couple; (3) selectively exposing the sensing surface to the redox couple during a measurement time period; and (4) performing an impedance measurement of the electrode during the measurement time period.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: (a) Schematic diagram of aptasensor operation, illustrating a partial blockage of electron transfer due to the formation of aptamer-target analyte complexes. (b) EIS responses to different concentrations of cortisol. (c) The sensor's corresponding calibration curve.

FIG. 2: (a) Intermittent impedance measurements (Nyquist plots) with about 10 min intervals, illustrating preserved stability of a sensing interface with the use of a protective layer. (b) Comparison of protected versus unprotected sensor response for about 5 nM concentration: the response of the unprotected sensing interface degrades over time. (c) Comparison of protected versus unprotected sensor calibration curve, illustrating a lowered sensitivity of the unprotected sensing interface.

FIG. 3: (a) Continuous sensor response characterization for progressively increasing cortisol concentration. (b) Aptasensor interference evaluation: comparison of sensor response to about 5 nM cortisol solution, to that with added interfering non-target analytes, including glucose (about 100 μM), lactate (about 100 μM) and progesterone (about 5 nM) (all spiked in Tris buffer).

FIG. 4: (a) Diurnal sweat cortisol profile of a healthy subject, as measured by a developed aptasensor. (b) Correlation of gold standard High Performance Liquid Chromatography (HPLC) and the aptasensor, demonstrating the sensor's high level of accuracy.

FIG. 5: (a) Schematic of a wearable device. (b) Schematic of a working electrode included in the wearable device.

### DETAILED DESCRIPTION

Wearable sweat analysis offers a desirable pathway toward non-invasive and continuous monitoring of hormones for a diverse set of clinical and personalized health monitoring applications. While sweat sensors demonstrate on-body measurement of a panel of target analytes, because of the lack of suitable in-situ signal enhancement strategies, their detection sensitivity remained in the μM to mM range. To overcome this barrier, here, some embodiments devise and combine two synergistic sensor development strategies: 1) direct electrochemical detection of capture probe-target analyte based on DNA aptamer-target analyte interactions using EIS and 2) incorporation of a redox couple-treated protective layer which enhances the sensitivity of EIS measurements, while mitigating against sensor surface degradation caused by the etching effect of the redox couple. As a demonstration, the methodology is applied for sensitive and on-body detection of cortisol, which is present in biofluids in nM levels (including sweat) and has clinical utility as a key metabolic regulator and a stress marker.

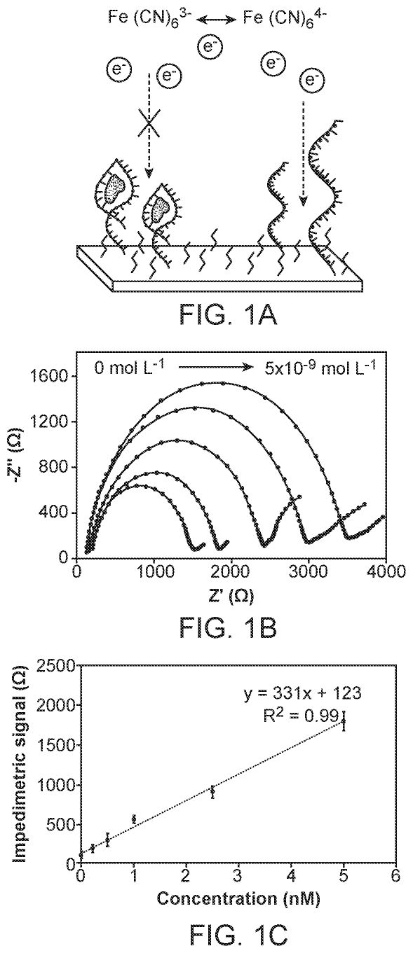

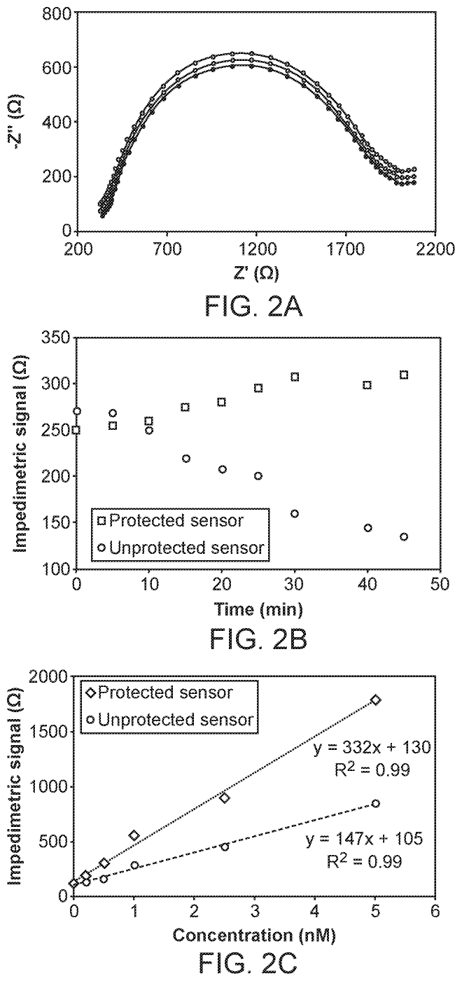

In the methodology of some embodiments, the underlying EIS-based sensing mechanism relies on monitoring changes to a working electrode's interfacial resistance, which is measured in the presence of a reversible Ferricyanide/Ferrocyanide redox couple [Fe(CN)₆]³⁻/[Fe(CN)₆]⁴⁻. A sensing interface includes cortisol-recognizing DNA aptamers, which are covalently immobilized on a surface of a working electrode. The formation of aptamer-cortisol complexes at the surface results in the partial blockage of electron-transfer (as shown in FIG. 1a). This leads to an increase in the interfacial resistance, which is correlated to a concentration of cortisol (corresponding measurement results are shown in FIG. 1b,c). Additionally, to overcome the etching effect of the redox couple, the redox couple is incorporated into a protective layer mounted on the sensor surface. Here, the etching effect is induced by cyanide ions (CN⁻) released from the [Fe(CN)₆]³⁻/[Fe(CN)₆]⁴⁻ redox couple, which otherwise can lead to gradual removal of the self-assembled monolayer of DNA probes. The incorporated protective layer preserves the sensing surface from direct exposure to a CN⁻-containing medium, while facilitating the signal transduction mechanism (FIG. 2a, b, c).

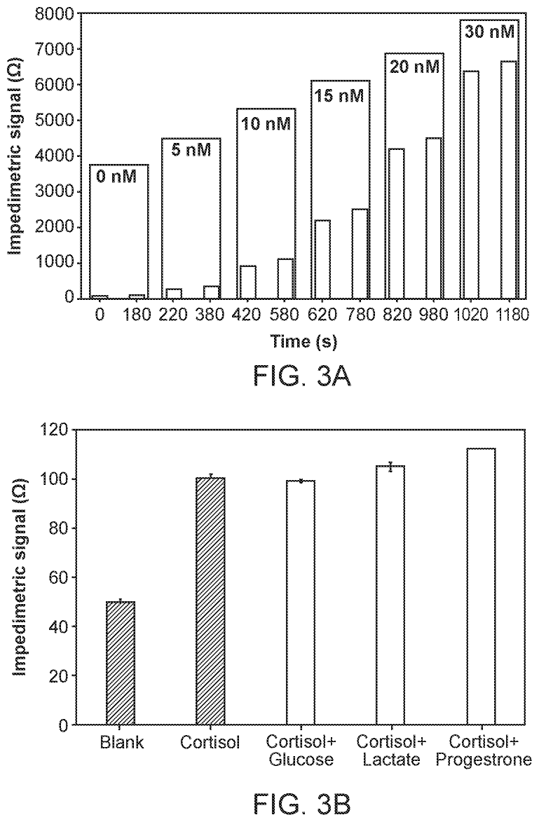

To characterize a sensor, first, a continuous response of the sensor for different cortisol concentration regimes (about 5 nM, about 10 nM, about 15 nM, and about 20 nM) is evaluated over time. As shown in FIG. 3a, the results indicate the preserved stability of the sensor's progressive response. Additionally, interference evaluation is performed to characterize the sensor's selectivity to ensure that non-target analytes in sweat (some with several orders of magnitude of higher concentration) do not interfere with the sensor's response. To this end, evaluation is made of the sensor response against an illustrative panel of non-target analytes including glucose, lactate, and progesterone (within their physiologically relevant range of concentrations). The obtained data show that non-specific bindings have minimal influence on the sensor response (as evident from small changes in the transduced signal, FIG. 3b).

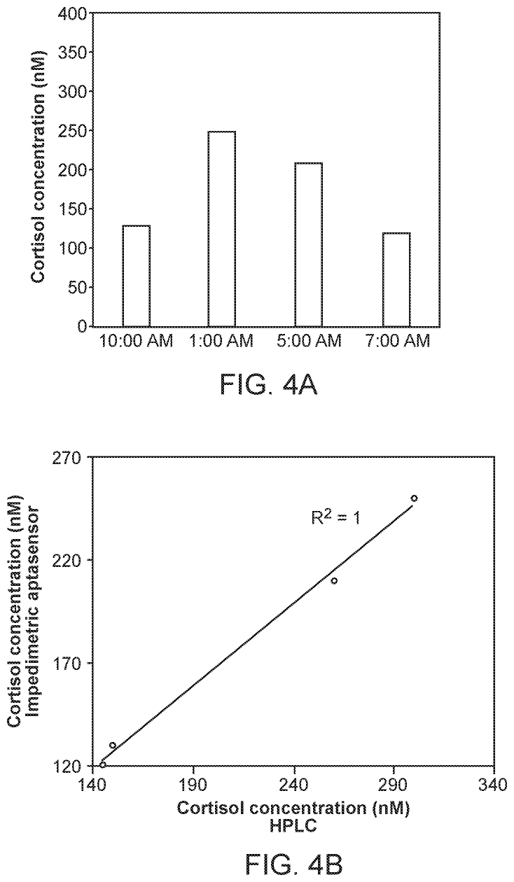

To demonstrate its applicability for clinical applications, the devised methodology is applied to analyze cortisol content of iontophoretically-induced sweat samples, collected from a healthy subject during a day (FIG. 4a). Sensor accuracy is validated by comparing concentration values, as estimated by the sensor response, with those obtained from High Performance Liquid Chromatography (HPLC), as a gold standard technique (FIG. 4b). The high degree of agreement for obtained measurements (R²=about 1) demonstrates the sensor's high level of accuracy. The versatility of the methodology allows for its use, with minimal effort and reconfiguration, to target various other hormones and metabolites in other non/minimally-invasive biofluids such as saliva, interstitial fluid, and urine. Therefore, the methodology can open directions for personalized health monitoring.

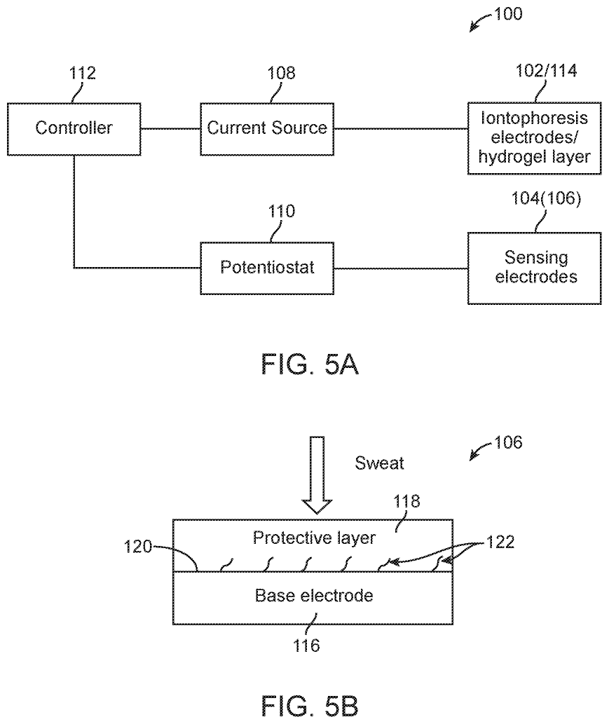

FIG. 5(a) is a schematic illustration of a wearable device 100 for sweat analysis according to some embodiments. The wearable device 100 includes a pair of iontophoresis electrodes 102/hydrogel layer 114 for sweat induction, and a set of sweat analyte sensing electrodes 104. The hydrogel layer 114 is adjacent to the iontophoresis electrodes 102, and the iontophoresis electrodes 102 are configured to interface a skin with the hydrogel layer 114 in between. The hydrogel layer 114 includes a secretory agonist (e.g., a cholinergic sweat gland secretory stimulating compound, such as pilocarpine), which is released when an electrical current is applied to the iontophoresis electrodes 102. The sensing electrodes 104 are configured to sense a target analyte, according to impedance measurements that are responsive to a presence or a concentration of the target analyte in induced sweat. The sensing electrodes 104 include a working electrode 106, a counter electrode, and, optionally, a reference electrode. Further details of the working electrode 106 are explained in connection with FIG. 5(b).

The wearable device 100 also includes a current source 108, which is connected to the iontophoresis electrodes 102 to activate sweat induction. A potentiostat 110 is also included in the wearable device 100, and is connected to the sensing electrodes 104 to obtain impedance measurements from the sensing electrodes 104. A controller 112 (e.g., including a processor and an associated memory storing processor-executable instructions) is also included in the wearable device 100, and is configured to control operation of various components of the wearable device 100. In particular, the controller 112 is configured to direct operation of the iontophoresis electrodes 102, through control of the current source 108, and to direct operation of the sensing electrodes 104, through control of the potentiostat 110. In addition, the controller 112 is configured to identify a presence of the target analyte and derive concentration measurements of the target analyte according to the impedance measurements. Although not shown, a wireless transceiver also can be included to allow wireless communication between the wearable device 100 and an external electronic device, such as a portable electronic device or a remote computing device.

FIG. 5(b) is a schematic illustration of the working electrode 106 according to some embodiments. The working electrode 106 includes a base electrode 116, which can be formed of, or can include, a metal such as gold, and a protective layer 118 disposed on a sensing surface 120 of the base electrode 116. Capture probes 122, which recognize and bind to the target analyte, are immobilized on the sensing surface 120 of the base electrode 116, such as through chemical bonding. The capture probes 122 can include DNA aptamers or other oligonucleotide or peptide aptamers. The protective layer 118 is implemented as an absorbent pad, which can be formed of, or can include, a polymeric material, a textile, or another absorbent material. The protective layer 118 includes a redox couple, such as [Fe(CN)₆]³⁻/[Fe(CN)₆]⁴⁻ or another combination of a reducing species and its corresponding oxidized form (e.g., Fe²⁺/Fe³⁺), that is incorporated within the protective layer 118. Incorporation of the redox couple can be performed by contacting the protective layer 118 with a solution of the redox couple (or another redox couple-containing liquid medium), followed by drying to remove a solvent. The sensing surface 120 of the base electrode 116 is configured to interface a skin with the protective layer 118 in between. Upon sweat induction, induced sweat from the skin reaches and flows through the protective layer 118, directing at least a portion of the redox couple towards the sensing surface 120 and the capture probes 122 to allow impedance measurements. Prior to and subsequent to sweat induction, the protective layer 118 reduces exposure of the sensing surface 120 to the redox couple and decouples (e.g., by spatially segregating) the sensing surface 120 from the redox couple, thereby mitigating against an etching effect of the redox couple during time periods when concentration measurements are not being performed. In place of, or in combination with, the protective layer 118, such selective exposure or coupling of the sensing surface 120 to the redox couple can be performed by another mechanism, such as by controllably directing flow of the redox couple through a valve.

Example Embodiments:

The following are example embodiments of this disclosure.

First Aspect

In some embodiments, a wearable device for biofluid analysis includes a set of sensing electrodes, and the set of sensing electrodes includes a working electrode which includes: (1) a base electrode including a sensing surface; (2) capture probes immobilized on the sensing surface; and (3) a protective layer disposed on the sensing surface and including a redox couple within the protective layer.

In any of the foregoing embodiments, the capture probes include aptamers.

In any of the foregoing embodiments, the aptamers include oligonucleotide aptamers.

In any of the foregoing embodiments, the capture probes are immobilized on the sensing surface via chemical bonding to the sensing surface.

In any of the foregoing embodiments, the capture probes are immobilized on the sensing surface via covalent bonding to the sensing surface.

In any of the foregoing embodiments, the protective layer includes an absorbent pad.

In any of the foregoing embodiments, the redox couple includes a reducing species and an oxidized form of the reducing species.

In any of the foregoing embodiments, the redox couple includes a Ferricyanide/Ferrocyanide redox couple.

In any of the foregoing embodiments, the device further includes a pair of iontophoresis electrodes and a secretory agonist-containing hydrogel layer adjacent to the pair of iontophoresis electrodes. In some embodiments, the device further includes a current source connected to the pair of iontophoresis electrodes. In some embodiments, the device further includes a potentiostat connected to the set of sensing electrodes. In some embodiments, the device further includes a controller connected to the potentiostat and the current source.

Second Aspect

In some embodiments, a method for biofluid analysis includes: (1) providing an electrode and capture probes immobilized on a sensing surface of the electrode; (2) providing a redox couple; (3) selectively exposing the sensing surface to the redox couple during a measurement time period; and (4) performing an impedance measurement of the electrode during the measurement time period.

In any of the foregoing embodiments, providing the redox couple includes spatially segregating the redox couple from the sensing surface during a time period prior to the measurement time period.

In any of the foregoing embodiments, providing the redox couple includes incorporating the redox couple in a protective layer, and disposing the protective layer on the sensing surface.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, a first numerical value can be “substantially” or “about” the same as a second numerical value if the first numerical value is within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

In the description of some embodiments, an object provided “on,” “over,” “on top of,” or “below” another object can encompass cases where the former object is directly adjoining (e.g., in physical or direct contact with) the latter object, as well as cases where one or more intervening objects are located between the former object and the latter obj ect.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

Some embodiments of this disclosure relate to a non-transitory computer-readable storage medium having computer code or instructions thereon for performing various processor-implemented operations. The term “computer-readable storage medium” is used to include any medium that is capable of storing or encoding a sequence of instructions or computer code for performing the operations, methodologies, and techniques described herein. The media and computer code may be those specially designed and constructed for the purposes of the embodiments of the disclosure, or they may be of the kind available to those having skill in the computer software arts. Examples of computer-readable storage media include volatile and non-volatile memory for storing information. Examples of memory include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like. Examples of computer code include machine code, such as produced by a compiler, and files containing higher-level code that are executed by a processor using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computing device via a transmission channel. Another embodiment of the disclosure may be implemented in hardwired circuitry in place of, or in combination with, processor-executable software instructions.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

1. A wearable device for biofluid analysis, comprising:
a set of sensing electrodes, the set of sensing electrodes including a working electrode which includes:
a base electrode including a sensing surface;
capture probes immobilized on the sensing surface; and
a protective layer disposed on the sensing surface and including a redox couple within the protective layer.

2. The wearable device of claim 1, wherein the capture probes include aptamers.

3. The wearable device of claim 2, where the aptamers include oligonucleotide aptamers.

4. The wearable device of claim 1, wherein the capture probes are immobilized on the sensing surface via chemical bonding to the sensing surface.

5. The wearable device of claim 4, wherein the capture probes are immobilized on the sensing surface via covalent bonding to the sensing surface.

6. The wearable device of claim 1, wherein the protective layer includes an absorbent pad.

7. The wearable device of claim 1, wherein the redox couple includes a reducing species and an oxidized form of the reducing species.

8. The wearable device of claim 1, wherein the redox couple includes a Ferricyanide/Ferrocyanide redox couple.

9. The wearable device of claim 1, further comprising a pair of iontophoresis electrodes and a secretory agonist-containing hydrogel layer adjacent to the pair of iontophoresis electrodes.

10. The wearable device of claim 9, further comprising a current source connected to the pair of iontophoresis electrodes.

11. The wearable device of claim 10, further comprising a potentiostat connected to the set of sensing electrodes.

12. The wearable device of claim 11, further comprising a controller connected to the potentiostat and the current source.

13. A method for biofluid analysis, comprising:
providing an electrode and capture probes immobilized on a sensing surface of the electrode;
providing a redox couple;
selectively exposing the sensing surface to the redox couple during a measurement time period; and
performing an impedance measurement of the electrode during the measurement time period.

14. The method of claim 13, wherein providing the redox couple includes spatially segregating the redox couple from the sensing surface during a time period prior to the measurement time period.

15. The method of claim 13, wherein providing the redox couple includes incorporating the redox couple in a protective layer, and disposing the protective layer on the sensing surface.
